(19) United States
(12) Patent Application Publication
(10) Pub. No.: US 2019/0209817 A1
(43) Pub. Date: Jul. 11, 2019

(54) HIGH FREQUENCY AND MEDICINE TREATMENT DEVICE FOR GENERATING CONTINUOUS SKIN IMPROVEMENT EFFECT

(71) Applicant: AGNES MEDICAL CO.,LTD, Seongnam-si (KR)

(72) Inventor: Gun-Young AHN, Seongnam-si (KR)

(73) Assignee: AGNES MEDICAL CO.,LTD, Seongnam-si (KR)

(21) Appl. No.: 16/205,496

(22) Filed: Nov. 30, 2018

(30) Foreign Application Priority Data
Jan. 9, 2018 (KR) 10-2018-0002767

(51) Int. Cl. A61M 37/00; A61N 1/06; A61N 1/28; A61N 1/30

(52) U.S. Cl. CPC A61M 37/00; A61N 1/06; A61N 1/28; A61M 2205/3653; A61M 2037/0007; A61M 2037/003; A61M 2205/3606; A61N 1/306

(57) ABSTRACT

Disclosed is a treatment device capable of suppressing generation of pain in a person to be treated and obtaining a continuous and fundamental skin treatment effect. The treatment device includes a handpiece body, a needle unit which is provided at one side of the handpiece body and includes a plurality of needles including hollow portions therein and having front ends inserted into an epidermis of skin of a person to be treated, a high frequency generator which is electrically connected to the needle unit, receives a high frequency current when the front ends of the needles are inserted into the epidermis, and heats the needles using the high frequency current, and a medicine delivery unit which communicates with the hollow portions of the plurality of needles and delivers a medicine to the epidermis through the hollow portions when the front ends of the needles are inserted into the epidermis.

### CROSS-REFERENCE TO RELATED APPLICATION

This application claims priority to and the benefit of Korean Patent Application No. 10-2018-0002767 filed on Jan. 9, 2018, the disclosure of which is incorporated herein by reference in its entirety.

### BACKGROUND

### 1. Field of the Invention

The present invention relates to a treatment device, and more specifically, to a treatment device capable of suppressing generation of pain in a person to be treated and obtaining a continuous and fundamental skin treatment effect by using both a high frequency current and a medicine.

### 2. Discussion of Related Art

In general, a high frequency current refers to an alternating current of 100,000 Hz or more. The high frequency current is characterized in that an electrochemical reaction or electrolysis phenomenon is not caused due to a short oscillation width, and oscillating current energy is converted into thermal energy only in a predetermined path.

In particular, when the high frequency current flows through a human tissue, the high frequency current hardly causes ion motion and does not cause an electrochemical reaction or electrolysis phenomenon due to a very short oscillation width. When high frequency electric energy is applied to a tissue, molecules constituting the tissue vibrate and rub against each other to generate bio-heat whenever a direction of a current is changed.

In addition, unlike other types of currents, the high frequency current does not stimulate a sensory nerve and a motor nerve, does not cause discomfort or muscle contraction in a human body, and generates heat energy. The heat energy may also induce additional effects of improving cell function and increasing blood flow.

Therefore, a high frequency treatment device is used for various treatments such as skin regeneration, lifting, suppression of secretion of a melanin pigment, obesity treatment, skin care, hair growth, and pain relief by using the high frequency current.

A general high frequency treatment device includes a handpiece including a high frequency electrode which comes into contact with a treatment region of skin and through which a high frequency current is applied and includes a body including a high frequency generator configured to receive power and supply a high frequency current to the handpiece and including a high frequency operation control unit configured to control operation of the high frequency generator, wherein the handpiece is configured to be electrically connected to the body through a cable or the like.

Such a general high frequency treatment device may cause a small amount of pain to a person to be treated. It is slightly difficult to continuously and fundamentally treat skin only through a treatment using the high frequency treatment device.

### PRIOR ART DOCUMENTS

### Patent Documents

Korea Patent Publication No. 2010-0136245 (Dec. 28, 2010: AUTOMATIC INJECTION GUN)

Korean Patent No. 10-1749702 (Jun. 15, 2017: HIGH FREQUENCY TREATMENT SYSTEM AND WEARABLE HIGH FREQUENCY GENERATOR FOR HIGH FREQUENCY TREATMENT SYSTEM)

### SUMMARY

The present invention has been made in view of the above matters and is directed to providing a treatment device capable of suppressing generation of pain in a person to be treated and obtaining a continuous and fundamental skin treatment effect by using a high frequency.

A high frequency and medicine treatment device for generating a continuous skin improvement effect according to embodiments of the present invention includes a handpiece body; a needle unit which is provided at one side of the handpiece body and includes a plurality of needles including hollow portions therein and having front ends inserted into an epidermis of skin of a person to be treated; a high frequency generator which is electrically connected to the needle unit, receives a high frequency current when the front ends of the needles are inserted into the epidermis, and heats the needles using the high frequency current; and a medicine delivery unit which communicates with the hollow portions of the plurality of needles and delivers a medicine to the epidermis through the hollow portions when the front ends of the needles are inserted into the epidermis.

### BRIEF DESCRIPTION OF THE DRAWINGS

The above and other objects, features and advantages of the present invention will become more apparent to those of ordinary skill in the art by describing exemplary embodiments thereof in detail with reference to the accompanying drawings, in which:

FIG. 1 is a view illustrating an overall configuration of a high frequency and medicine treatment system for generating a continuous skin improvement effect according to an embodiment of the present invention;

FIG. 2 is a perspective view illustrating a high frequency and medicine treatment device for generating a continuous skin improvement effect according to an embodiment of the present invention;

FIG. 3 is a reference view illustrating a skin treatment sequence of the high frequency and medicine treatment device for generating the continuous skin improvement effect according to the embodiment of the present invention; and

FIG. 4 is a configuration view illustrating an internal configuration of the high frequency and medicine treatment device for generating the continuous skin improvement effect according to the embodiment of the present invention.

### DETAILED DESCRIPTION

Before embodiments of the invention are described in detail, it should be understood that the present invention is not limited to components set forth in the following description or illustrated in the following drawings. The present invention may be practiced or conducted in various ways.

Also, it is to be understood that the phraseology and terminology used herein is for the purpose of description and should not be regarded as limiting.

Unless specified or limited otherwise, the terms “mounted,” “installed,” “connected,” “supported,” and “coupled” and variations thereof are used broadly and encompass both direct and indirect mountings, installations, connections, supports, and couplings. Further, “connected” and “coupled” are not restricted to physical or mechanical connections or couplings.

Although directional references, such as upper, lower, downward, upward, rearward, bottom, front, rear, etc., may be made herein in describing the drawings, these references are made relative to the drawings (as normally viewed) for convenience. These directions are not intended to be taken literally or limit the present invention in any form.

In addition, terms such as “first”, “second”, and “third” are used herein for purposes of description and are not intended to indicate or imply relative importance or significance.

Hereinafter, embodiments of the present invention will be described in detail with reference to the accompanying drawings.

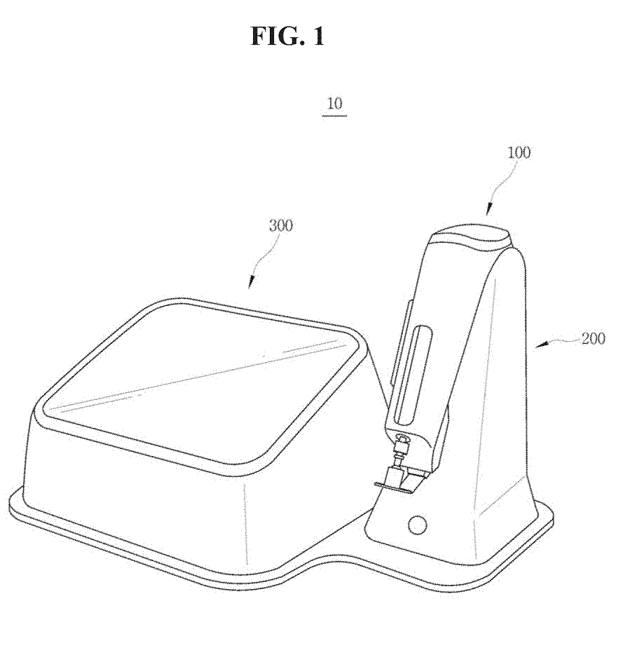

FIG. 1 is a view illustrating an overall configuration of a high frequency and medicine treatment system 10 (hereinafter referred to as a “treatment system”) for generating a continuous skin improvement effect according to an embodiment of the present invention. The treatment system 10 according to the embodiment of the present invention mainly includes a high frequency and medicine treatment device 100 (hereinafter referred to as a “treatment device”) configured to generate a continuous skin improvement effect, a treatment device control unit 300, and a treatment device support unit 200.

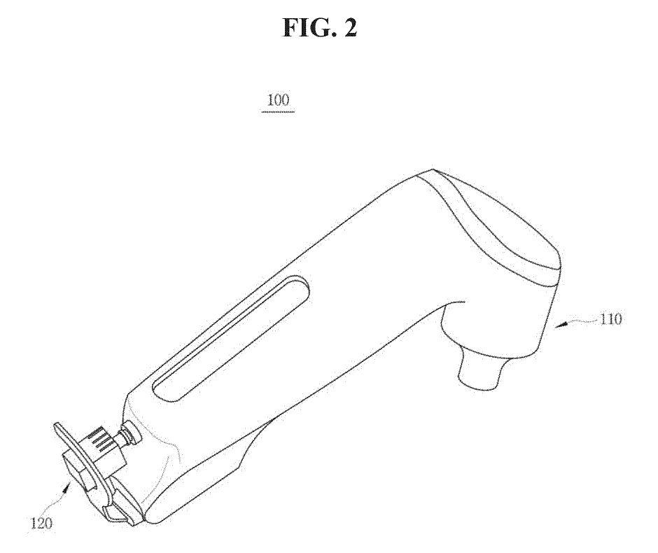

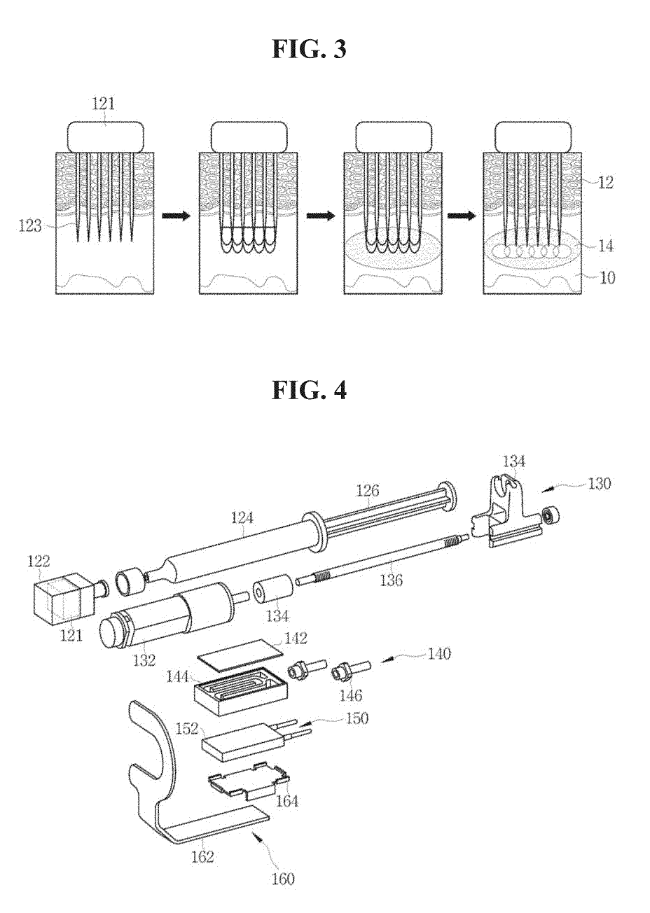

The treatment device 100 will be described in detail with reference to FIGS. 2 and 3. The treatment device control unit 300 is a device configured to provide a status information guide to the treatment device 100 or allow a user to set an operation of the treatment device 100. The treatment device control unit 300 is provided in the form of a touch panel display, variously informs the user of a power source, an operation mode, and the like of the treatment device 100, and allows the user to change the operation mode and set the treatment device 100.

Next, the treatment device support unit 200 is provided to mount the treatment device 100 thereon and charge the power source of the treatment device 100. Alternatively, the treatment device support unit 200 includes a separate portion at one side thereof to disinfect the treatment device 100 or is provided to store a needle unit 120 and a medicine delivery unit 130 which are interchangeably provided in the treatment device 100.

FIG. 2 is a perspective view illustrating a treatment device 100 according to an embodiment of the present invention. The treatment device 100 includes a handpiece body 110, a needle unit 120, a high frequency generator, a medicine delivery unit 130, and a skin pressing and expanding unit 160.

The handpiece body 110 is provided in a streamlined shape such that a user holds the treatment device 100 with a hand to easily perform a treatment on skin of a person to be treated. The handpiece body 110 includes a button unit at one side thereof, which is configured to control operation of the treatment device 100 to operate or stop the treatment device 100.

The treatment device 100 may be connected to the treatment device support unit 200 in a wired or wireless manner and may receive a high frequency current. Various sensors capable of measuring status information of the treatment device 100 are provided in the treatment device 100, and thus, status information data of the treatment device 100 may be transmitted to the treatment device control unit 300, or data on operation control of the treatment device 100 may be received from the treatment device control unit 300.

Next, the needle unit 120 includes a plurality of needles 123 having hollow portions therein and a needle support unit 121 which fixedly supports the plurality of needles 123, has a hollow portion therein which communicates with the hollow portions of the plurality of needles 123, and is detachably provided in the handpiece body 110. The needle unit 120 includes a separate detachable needle cap 122 configured to protect the needles 123 from external contamination or the like.

The needle support unit 121 is electrically connected to the handpiece body 110, and the hollow portion formed in the needle supporter 121 is configured to communicate with the medicine delivery unit 130.

The high frequency generator may supply a high frequency current to the needle support unit 121 and may be provided inside the handpiece body 110 or provided in the treatment device support unit 200 according to a user's choice.

The medicine delivery unit 130 communicates with the needle support unit 121 to deliver a medicine to an epidermis of skin of a person to be treated through the needles 123 inserted into the epidermis of the skin of the person to be treated. The medicine delivery unit 130 is provided in the form of a syringe inside the handpiece body 110.

That is, as shown in FIG. 4, the medicine delivery unit 130 includes a syringe body 124 into which a medicine is injected, a syringe piston 126 inserted into the syringe body 124 and configured to inject the medicine by applying pressure, a syringe supporter 134 configured to detachably support the syringe body 124 and the syringe piston 126, and motors 132, 134, and 136 configured to control the syringe piston 126 to be laterally moved.

The motors 132, 134, and 136 are configured to control generation of reverse pressure when a medicine is injected.

That is, high pressure is generated in the syringe body 124 when a medicine with high viscosity is injected at high pressure in a short time through the needles 123 with a narrow inner diameter. The high pressure in this syringe body 124 generates pressure (reverse pressure) in a direction opposite to movement of the syringe piston 126 pushing the medicine with the high viscosity. This reverse pressure is applied to a head portion of the syringe piston 120 to instantaneously compress the head portion made of a rubber material.

The compression causes a phenomenon in which the head portion is restored to an original state as time goes on and thus a medicine is slowly pushed to the outside by an amount corresponding to a restored volume. In order to prevent the phenomenon in which the medicine is slowly ejected due to the reverse pressure, the motors 132, 134, and 136 inject the medicine with a force as strong as a set value and then move the syringe piston 126 in a direction of the reverse pressure, i.e., a direction opposite to an injection direction to prevent the medicine from being ejected due to the reverse pressure.

Next, the skin pressing and expanding unit 160 expands a treatment layer of skin of the person to be treated to allow the needles 123 having no separate negative pressure structure to be completely inserted into an epidermis of the person to be treated. The skin pressing and expanding unit 160 supplies coolness to the treatment layer of skin of the person to be treated to cool a surface layer of skin and relieve pain associated with a treatment of the person to be treated.

To this end, as shown in FIG. 4, the skin pressing and expanding unit 160 includes a guide 162, a thermoelectric element 150 configured to supply coolness to the guide 162, and a coolant circulation module 140.

The guide 162 is provided in a horseshoe shape. When a horseshoe portion is placed and pressed on the skin of the person to be treated, a portion of a layer of skin protrudes inside the horseshoe portion, and thus, the skin layer expands.

When skin protrudes by a certain height as described above, the needles 123 may be inserted by a certain depth.

The guide 162 in contact with the skin layer supplies coolness to the skin surface layer of the person to be treated, thereby preventing a burn to skin due to a high frequency and relieving pain of the person to be treated.

Hereinafter, a skin treatment sequence and operation of the high frequency and medicine treatment device 100 for generating the continuous skin improvement effect according to the embodiment of the present invention will be described with reference to FIG. 3.

First, referring to the left portion of FIG. 3, when the needles 123 are inserted into the epidermis of the skin of the person to be treated and then a high frequency is applied, collagen of the epidermis of the skin is remodeled. When such a high frequency is applied, the epidermis of the skin receives a weak electric burn.

A medicine is delivered through the medicine delivery unit 130. As described above, when the collagen of the epidermis of the skin is modified through the high frequency, the epidermis of the skin receives a weak electric burn, and the medicine is delivered, duration of action of the injected medicine is increased, thereby improving skin elasticity and minimizing pores and fine wrinkles. The weak electric burn inside a skin tissue may inhibit absorption of a hyaluronic acid of the injected medicine, thereby increasing the duration of an effect in which skin looks clear due to the medicine.

In addition to such a treatment, coolness is transferred to an external part of a treatment area to relieve pain and prevent a burn to skin due to a high frequency.

According to a high frequency and medicine treatment device for generating a continuous skin improvement effect according to the present invention, collagen of an epidermis of skin can be remodeled using a high frequency, and then, a medicine can be injected to increase duration of action of the medicine, thereby improving a skin improvement effect.

In addition, according to a high frequency and medicine treatment device for generating a continuous skin improvement effect according to the present invention, a surface layer of a treatment skin of a person to be treated can be expanded and treated during a treatment, thereby further improving a treatment effect.

Furthermore, according to a high frequency and medicine treatment device for generating a continuous skin improvement effect according to the present invention, a surface layer of a treatment skin of a person to be treated can be cooled and treated during a treatment, thereby relieving pain of the person to be treated.

In addition, according to a high frequency and medicine treatment device for generating a continuous skin improvement effect according to the present invention, it is possible to remove reverse pressure generated when a medicine for a treatment is injected, thereby precisely controlling a medicine injection.

Although the exemplary embodiments of the present invention have been described hereinabove, various modifications and alterations and equivalents of the present invention are possible. The exemplary embodiments of the present invention may be appropriately modified and similarly applied. Therefore, contents described in the exemplary embodiments do not limit the scope of the present invention as defined by the claims.

What is claimed is:

1. A high frequency and medicine treatment device for generating a continuous skin improvement effect, the high frequency and medicine treatment device comprising:
a handpiece body;
a needle unit which is provided at one side of the handpiece body and includes a plurality of needles including hollow portions therein and having front ends inserted into an epidermis of skin of a person to be treated;
a high frequency generator which is electrically connected to the needle unit, receives a high frequency current when the front ends of the needles are inserted into the epidermis, and heats the needles using the high frequency current; and
a medicine delivery unit which communicates with the hollow portions of the plurality of needles and delivers a medicine to the epidermis through the hollow portions when the front ends of the needles are inserted into the epidermis.

2. The high frequency and medicine treatment device of claim 1, further comprising a skin pressing and expanding unit configured to expand a skin layer of the person to be treated by applying pressure to the skin layer into which the front ends of the plurality of needles are inserted.

3. The high frequency and medicine treatment device of claim 2, wherein, when the skin layer of the person to be treated is expanded, the skin pressing and expanding unit supplies coolness to the skin layer of the person to be treated, which comes into contact therewith, and cools the skin layer of a treatment skin of the person to be treated.

4. The high frequency and medicine treatment device of claim 1, wherein the needle unit and the medicine delivery unit are detachably provided.

5. The high frequency and medicine treatment device of claim 1, wherein the medicine delivery unit further includes a reverse pressure removal unit configured to remove reverse pressure generated when the medicine is delivered to the epidermis through the hollow portions of the needles.
